(19) United States
(12) Patent Application Publication
(10) Pub. No.: US 2015/0250380 A1
(43) Pub. Date: Sep. 10, 2015

(54) THREE-DIMENSIONAL ENDOSCOPE

(71) Applicant: OLYMPUS CORPORATION, Tokyo (JP)

(72) Inventor: Hiromu IKEDA, Tokyo (JP)

(73) Assignee: OLYMPUS CORPORATION, Tokyo (JP)

(21) Appl. No.: 14/716,293

(22) Filed: May 19, 2015

Related U.S. Application Data
(63) Continuation of application No. PCT/JP2013/085337, filed on Dec. 26, 2013.
(60) Provisional application No. 61/746,809, filed on Dec. 28, 2012.

(51) Int. Cl. A61B 1/00; A61B 1/04

(52) U.S. Cl. CPC A61B 1/00183; A61B 1/00193; A61B 1/00096; A61B 1/04; A61B 1/00009; A61B 1/00133; A61B 1/00006; A61B 1/00087

(57) ABSTRACT

This three-dimensional endoscope is provided with optical systems that are provided with a pair of lens groups, which focus light coming from an object, a pair of first prisms, which deflect the light focused by the respective lens groups, and a pair of second prisms, which deflect the light deflected by the respective first prisms, and that form two images exhibiting parallax; a distal-end-side rotating mechanism that rotates the pair of lens groups and the pair of first prisms relative to the pair of second prisms about an axis perpendicular to the optical axes; an imaging device that captures the two images exhibiting parallax formed by the optical systems; and an image processing portion that processes screen images acquired by the imaging device and that rotates the two images acquired by the imaging device in opposite directions from each other.

### CROSS-REFERENCE TO RELATED APPLICATIONS

This application is a continuation application of PCT/JP2013/085337 filed on Dec. 26, 2013 which claims priority to U.S. Provisional Patent Application No. 61/746,809 filed on Dec. 28, 2012, the contents of each of which is incorporated herein by reference.

### TECHNICAL FIELD

The present invention relates to a three-dimensional endoscope.

### BACKGROUND ART

In the related art, there is an endoscope imaging apparatus that is provided with a pair of negative lens groups, a pair of first positive lens groups, an aperture stop, a second positive lens group, and a single imaging device, which are sequentially disposed from the object side, wherein the optical axis of the second positive lens group is decentered with respect to the optical axes of the lens groups located closer to the object side than the aperture stop is (for example, see Patent Literature 1).

In this endoscope imaging apparatus, the pair of lens groups, the imaging device, and a drive circuit that is disposed immediately after the imaging device and that transmits an image acquired by the imaging device to an image processing device in the form of electrical signals are disposed at the distal end of an endoscope insert portion so that the pair of lens groups, the imaging device, and the drive circuit are operated as a single unit.

### CITATION LIST

### Patent Literature

### Patent Literature 1: Japanese Unexamined Patent Application, Publication No. 2001-147382

### DISCLOSURE OF INVENTION

An aspect of the present invention provides a three-dimensional endoscope including the following elements: an optical system that is provided with a pair of lens groups which focus light coming from an object and which have substantially parallel optical axes, a pair of first prisms which deflect rays of light focused by the respective lens groups by 90° in opposite directions, and a pair of second prisms which deflect the rays of light deflected by the respective first prisms by an additional 90° so as to make the rays of light parallel to each other, and thereby forming two images exhibiting parallax; a distal-end-side rotating mechanism that rotates, as a single unit, the pair of lens groups and the pair of first prisms of the optical system relative to the pair of second prisms about an axis perpendicular to the optical axes; an imaging device that captures at a single imaging surface the two images exhibiting parallax formed by the optical system; and an image processing portion that rotates the two images acquired by the imaging device in opposite directions from each other by an angle formed between a plane containing the optical axes of the pair of lens groups and an axis perpendicular to the imaging surface by means of processing the images acquired by the imaging device.

### BRIEF DESCRIPTION OF DRAWINGS

FIG. 1 is a diagram showing an overall configuration of a three-dimensional endoscope according to a first embodiment of the present invention.

FIG. 2 is a diagram showing an arrangement of an optical system and an imaging device of the three-dimensional endoscope in FIG. 1.

FIG. 3 is a diagram showing a state in which a distal end portion of the three-dimensional endoscope in FIG. 1 is not pivoted and showing (a) the arrangement of the optical system and (b) an example screen image.

FIG. 4 is a diagram showing a state in which the distal end portion of the three-dimensional endoscope in FIG. 1 is pivoted 30° and showing (a) the arrangement of the optical system and (b) an example screen image.

FIG. 5 is a diagram showing a state in which a distal end portion of the three-dimensional endoscope in FIG. 1 is pivoted 90° and showing (a) the arrangement of the optical system and (b) an example screen image.

FIG. 6 is a diagram showing a modification of the three-dimensional endoscope in FIG. 1.

FIG. 7 is a diagram showing an overall configuration of a three-dimensional endoscope according to a second embodiment of the present invention.

FIG. 8 is a diagram individually showing the three-dimensional endoscope in FIGS. 7 (a) in a state in which a distal end portion and an intermediate portion thereof are extended straight and (b) in a state in which the distal end portion and the intermediate portion are pivoted in an interlinked fashion in opposite directions from each other.

FIG. 9 is a diagram showing a modification of an interlinking mechanism of the three-dimensional endoscope in FIG. 7, individually showing (a) a state in which a distal end portion and an intermediate portion thereof are extended straight, (b) a state in which the distal end portion and the intermediate portion are pivoted in an interlinked fashion in opposite directions from each other, and (c) a state in which the distal end portion alone is independently pivoted.

FIG. 10 is a diagram showing a modification of the three-dimensional endoscope in FIG. 7 having a moving mechanism, individually showing (a) a state in which a distal end portion and an intermediate portion thereof are extended straight, (b) a state in which the distal end portion and the intermediate portion are pivoted in an interlinked fashion in opposite directions from each other and are moved forward by the moving mechanism, and (c) a state in which the distal end portion and the intermediate portion are pivoted in an interlinked fashion further in opposite directions from each other and are moved forward further by the moving mechanism.

FIG. 11 is a diagram individually showing (a) an example screen image for the case shown in FIG. 10(a), and (b) an example screen image for the case shown in FIG. 10(c).

### EMBODIMENT FOR CARRYING OUT THE INVENTION

A three-dimensional endoscope 1 according to a first embodiment of the present invention will be described below with reference to the drawings.

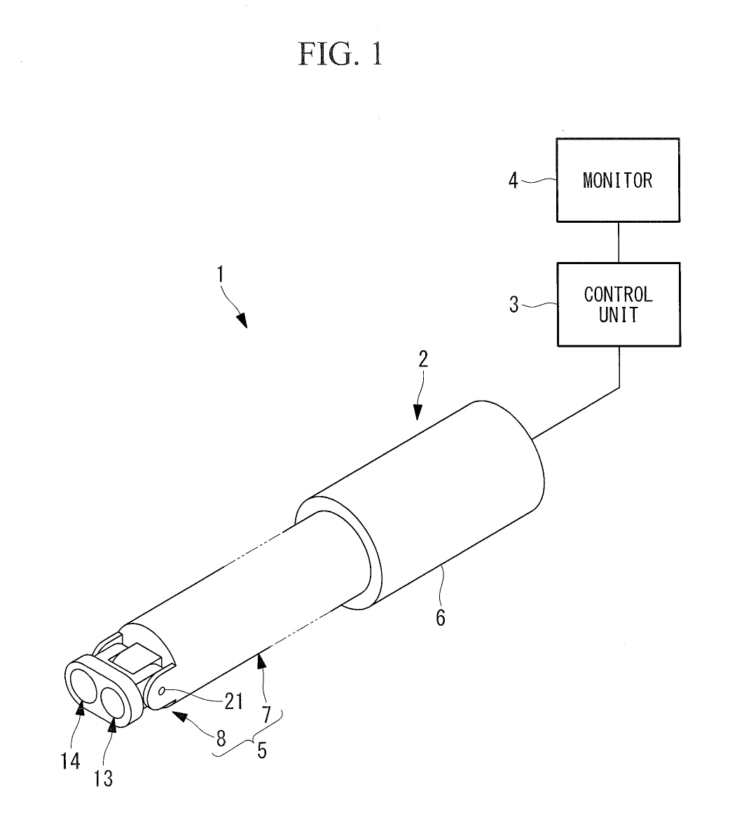

As shown in FIG. 1, the three-dimensional endoscope 1 according to this embodiment is provided with an endoscope main unit 2, a control unit 3 that is connected to the endoscope main unit 2, and a monitor 4 that displays an screen image acquired by the endoscope main unit 2.

The endoscope main unit 2 is provided with a long insert portion 5 and a manipulation portion 6 that is disposed at the base end side of the insert portion 5.

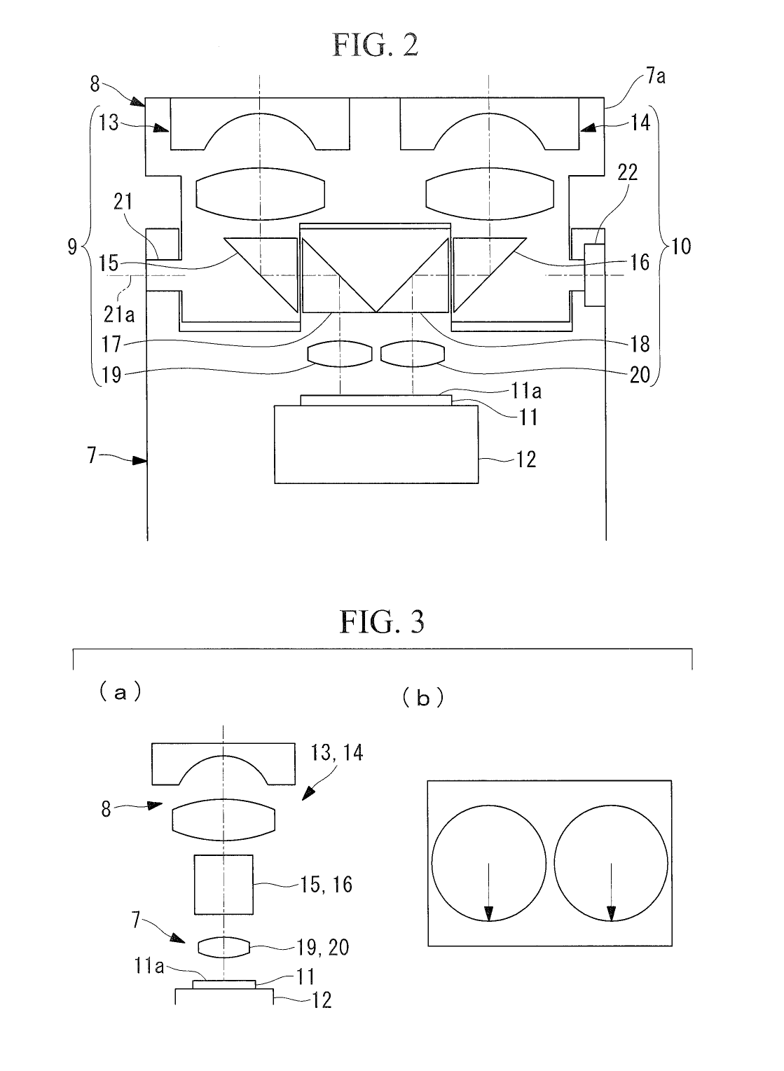

The insert portion 5 is provided with a long proximal end portion 7 and a distal end portion 8 that is provided at the distal end of the proximal end portion 7 so as to be pivotable about an axis perpendicular to the longitudinal axis thereof. As shown in FIG. 2, at the interior of the distal end portion 8 and the proximal end portion 7, a pair of optical systems 9 and 10 are disposed, and an imaging device 11, such as a CCD, that captures light focused by the optical systems 9 and 10 and a control circuit 12 that controls the imaging device 11 are also provided.

The pair of optical systems 9 and 10 are provided with a pair of lens groups 13 and 14 and a pair of first prisms 15 and 16, which are disposed in the distal end portion 7, as well as a pair of second prisms 17 and 18 and a pair of focusing lenses 19 and 20 that are disposed in the proximal end portion 7.

The distal end portion 8 is provided with a casing 7a that is pivotably attached to the distal end of the cylindrical proximal end portion 7, and the pair of lens groups 13 and 14 and the pair of first prisms 15 and 16 are accommodated in the casing 7a.

The pair of lens groups 13 and 14 have substantially parallel optical axes and are configured so as to focus light coming from an object.

Each of the first prisms 15 and 16 is individually disposed at the base end side of each of the lens groups 13 and 14 and is configured so as to deflect beams focused by the lens groups 13 and 14 by 90°, thus directing the beams radially inward along the center axis 21a of a pivoting shaft 21 of the casing 7a.

In addition, the distal end portion 8 is driven by a motor (distal-end-side rotating mechanism) 22 so as to be pivoted about the above-described pivoting axis relative to the proximal end portion 7.

The pair of second prisms 17 and 18 disposed in the proximal end portion 7 are disposed side-by-side in a direction parallel to the center axis 21a of the pivoting shaft 21 of the casing 7a at positions facing the pair of first prisms 15 and 16, respectively, and are configured so as to deflect the beams deflected by the first prisms 15 and 16 by an additional 90°, thus directing them in the direction toward the base end of the proximal end portion 7 along the longitudinal direction thereof.

The pair of focusing lenses 19 and 20 respectively focus the light deflected by the pair of second prisms 17 and 18 so as to form images at an imaging surface 11a of the imaging device 11.

The imaging device 11 has the imaging surface 11a at which the two images of the object formed by the pair of focusing lenses 19 and 20 are formed side-by-side at the same time.

Signals acquired by the imaging device 11 are converted to image information at the control circuit 12 and are transmitted to the control unit 3.

The control unit 3 controls the motor 22 and sets the angle by which the distal end portion 8 is pivoted relative to the proximal end portion 7.

In addition, the control unit 3 is configured so as to generate screen images showing the two images of the object by processing the image information transmitted thereto from the control circuit 12, and also so as to generate a screen image that can be perceived as a three-dimensional image by processing the two generated screen images based on the pivoting angle and to output it to the monitor 4.

Here, the relationship between the pivoting angle of the distal end portion 8 and the screen images acquired by the imaging device 11 will be described.

As shown in FIG. 3(a), in the state in which a plane containing the optical axes of the pair of lens groups 13 and 14 is parallel to the normal line of the imaging surface 11a, the two images in the screen images acquired by the imaging device 11 are directed to the same direction, as shown in FIG. 3(b). Therefore, the two screen images generated individually based on these two images are disposed so as to allow the brain to combine the screen images into a three-dimensional image of the object when separately viewed with the left and right eyes without changing the angles thereof.

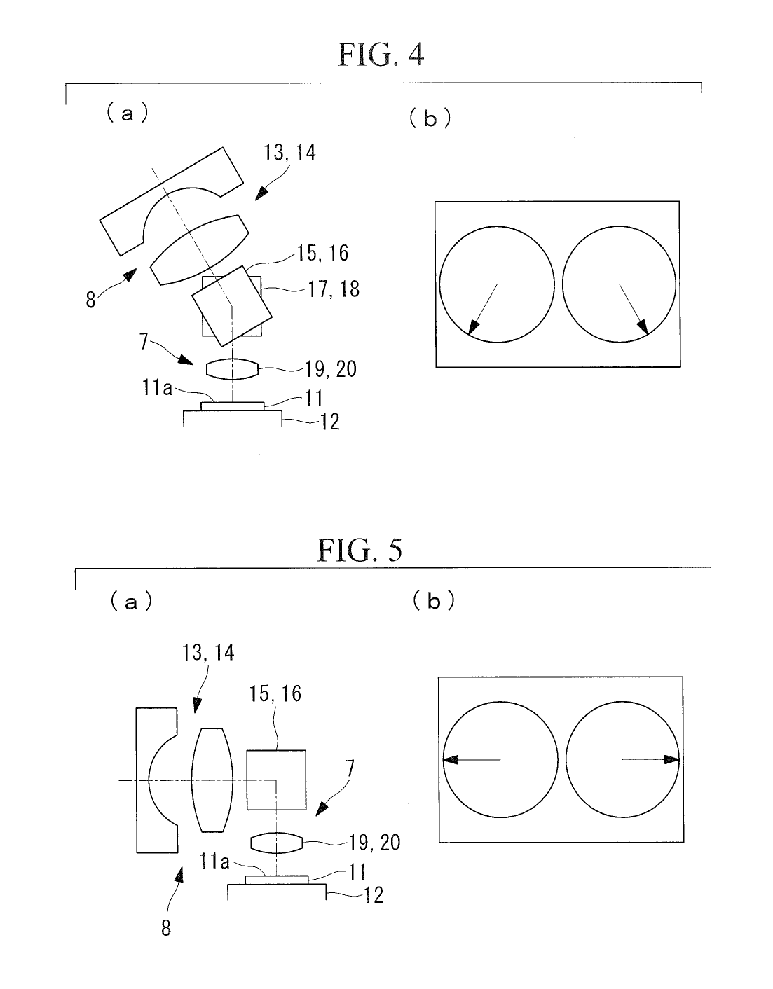

In contrast, when the distal end portion 8 is pivoted relative to the proximal end portion 7 by means of the control unit 3, thus changing the angles formed between the plane containing the optical axes of the pair of lens groups 13 and 14 and the normal line of the imaging surface 11a, as shown in FIG. 4(a) and FIG. 5(a), the two images in the screen images acquired by the imaging device 11 are rotated in opposite directions from each other by angles equal to the pivoting angles (30° and 90°) of the distal end portion 8, as shown in FIG. 4(b) and FIG. 5(b). When two screen images are generated individually based on these images, if no modification is applied, a screen image that cannot be perceived as a three-dimensional image will be obtained.

Therefore, the control unit 8 performs image processing in which the two generated screen images are rotated based on the pivoting angles of the distal end portion 8 in directions which are opposite to the directions in which the screen images pivot so as to bring the two images back to the same positions as those shown in FIG. 3(b).

The operation of the thus-configured three-dimensional endoscope 1 according to this embodiment will be described below.

With the three-dimensional endoscope 1 according to this embodiment, when the insert portion 5 is inserted into the body of a patient and the imaging device 11 is operated, two images acquired by the pair of optical systems 9 and 10 are formed at the imaging surface 11a of the imaging device 11 at the same time. Because the two lens groups 13 and 14 are disposed parallel to each other and with a space therebetween, the acquired images exhibit parallax, which allows the brain to combine them into a three-dimensional image of an object when separately viewed with the left and right eyes.

Then, when the observation direction needs to be changed, by pivoting the distal end portion 8 relative to the proximal end portion 7 by operating the motor 22 by means of the control unit 3, the optical axes of the lens groups 13 and 14 are pivoted.

In this case, in comparison with a conventional three-dimensional endoscope in which a distal end portion thereof accommodating an optical system, an imaging device, and a control circuit as a single unit is pivoted, with the three-dimensional endoscope 1 according to this embodiment, because only portions of the optical systems 9 and 10 are accommodated in the distal end portion 7, there is an advantage of enabling the endoscope to decrease the interference with other tissue or the like in a small space in the body by reducing the rotation radius by means of decreasing the length of the distal end portion 7, and it is possible to easily change the observation direction.

Furthermore, with the three-dimensional endoscope 1 according to this embodiment, because the two prisms 15 and 17 (16 and 18) deflect a beam into a crank shape at an intermediate portion thereof and the relative angle between the two prisms 15 and 17 (16 and 18) is changed by pivoting the distal end portion 8, two images formed at the imaging surface 11a are individually rotated in opposite directions by an angle equal to the pivoting angle of the distal end portion 8. With the three-dimensional endoscope 1 according to this embodiment, by subjecting screen images, which include the two images acquired due to such a rotation, to the image processing at the control unit 3, corrections are made so that the directions of the two screen images become the same; therefore, the two generated screen images exhibiting parallax can be perceived as a three-dimensional image of the object.

Note that, in this embodiment, although the motor 22 has been described as an example of the distal-end-side rotating mechanism that pivots the distal end portion 8 relative to the proximal end portion 7, another arbitrary rotating mechanism such as a wire, a linkage, and so forth may be employed.

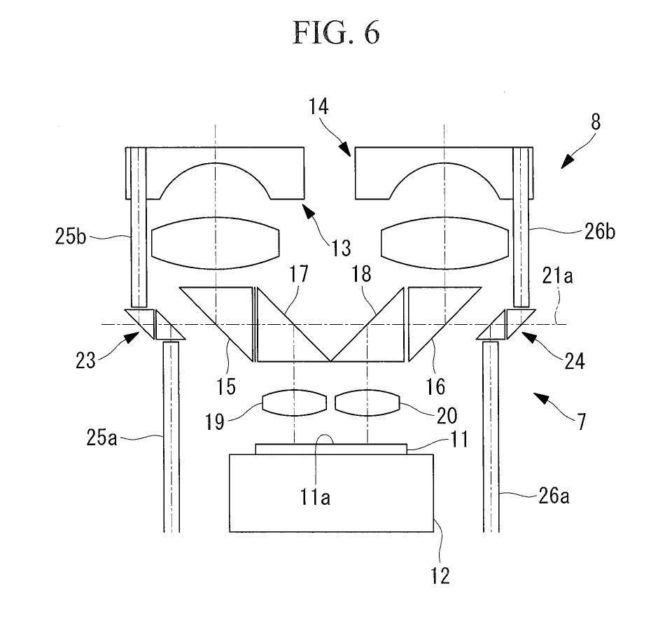

In addition, as shown in FIG. 6, in the case in which illumination light is made to exit from the distal end of the distal end portion 8 by means of a light guide, prism pairs 23 and 24 that can be relatively rotated about the pivoting axis 21a, which is same as the pivoting axis 21a of the distal end portion 8, may be provided and the light may be guided to light guides 25b and 26b of the distal end portion 8 from light guides 25a and 26a of the proximal end portion 7 so that a region to be captured is always illuminated with the same light level.

Next, a three-dimensional endoscope 30 according to a second embodiment of the present invention will be described with reference to the drawings.

In describing the three-dimensional endoscope 30 according to this embodiment, the same reference signs are assigned to portions which have the same configurations as those of the three-dimensional endoscope 1 according to the first embodiment described above, and descriptions thereof will be omitted.

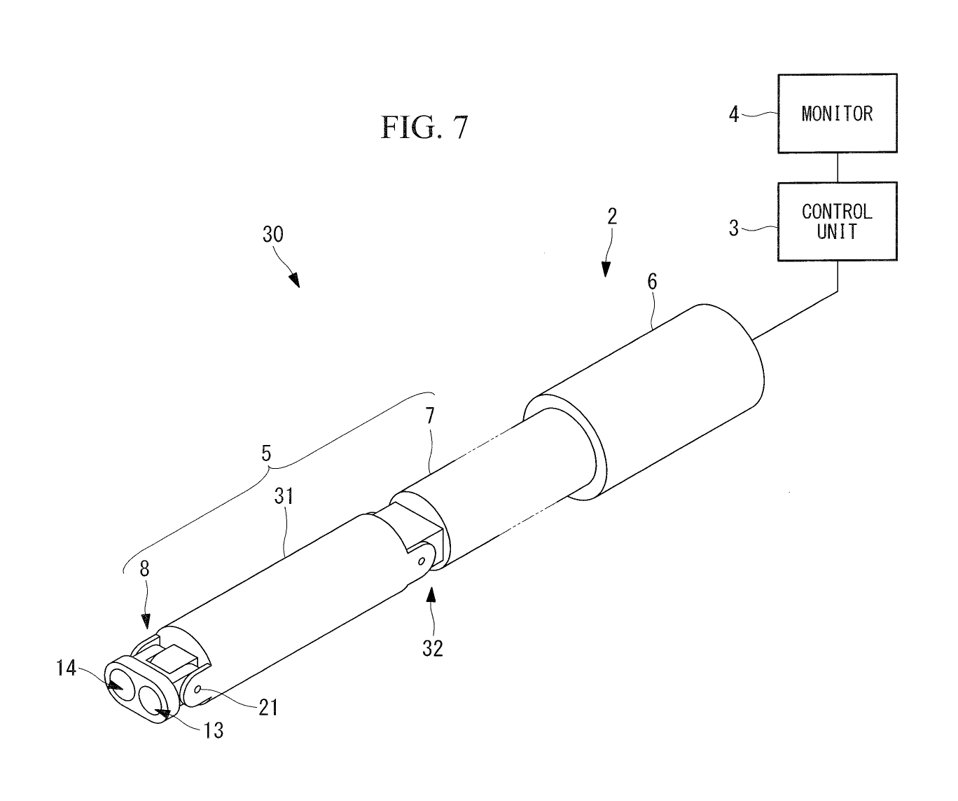

As shown in FIG. 7, a three-dimensional endoscope 30 according to this embodiment differs from the three-dimensional endoscope 1 according to the first embodiment in that an intermediate portion 31 is provided between the proximal end portion 7 and the distal end portion 8 and that a base-end-side rotating mechanism 32 that pivots the intermediate portion 31 relative to the proximal end portion 7 is provided. The base-end-side rotating mechanism 32 is also, for example, a motor.

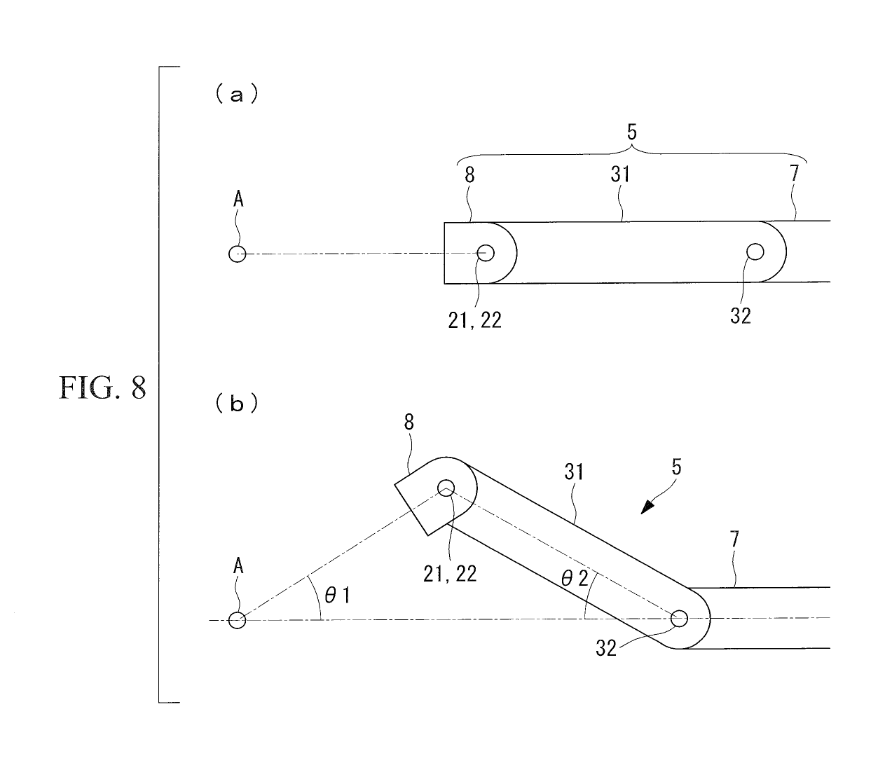

In addition, in the three-dimensional endoscope 30 according to this embodiment, the control unit 3 constitutes an interlinking mechanism that causes the motor (distal-end-side rotating mechanism) 22 and the motor (base-end-side rotating mechanism) 32 to move in an interlinked fashion. Specifically, as shown in FIG. 8(b), the control unit 3 is configured so as to operate the two motors 22 and 32 in an interlinked fashion so that an angle θ1 formed between the plane containing the optical axes of the pair of lens groups 13 and 14 of the distal end portion 8 and the longitudinal axis of the proximal end portion 7 and an angle θ2 formed between the longitudinal axis of the intermediate portion 31 and the longitudinal axis of the proximal end portion 7 are always changed with a constant proportion.

In examples shown in FIGS. 8(a) and (b), the two motors 22 and 32 are configured so as to be rotated in opposite directions from each other.

The operation of the thus-configured three-dimensional endoscope 30 according to this embodiment will be described below.

To observe a treatment target in a body by using the three-dimensional endoscope 30 according to this embodiment, rotating the two motors 22 and 32 in the interlinked fashion by means of the control unit 3, pivoting of the distal end portion 8 relative to the intermediate portion 31 and pivoting of the intermediate portion 31 relative to the proximal end portion 7 are performed in opposite directions, and thus, the insert portion 5 as a whole takes a substantially S-shape form.

By doing so, the same treatment target A can be observed by changing the angles. In this case, with the three-dimensional endoscope 30 according to this embodiment, because the length of the distal end portion 8 is short, there is an advantage in that the amount of protrusion in a direction that intersects the longitudinal axis direction of the proximal end portion 7 when angles of the distal end portion 8 is changed can be kept low. Therefore, it is possible to easily observe the treatment target A from various angles even in a small space in the body.

In addition, for example, in the case in which the treatment target A is treated with a treatment tool protruding from the proximal end portion 7, because observation angles can be changed without changing the orientation of the proximal end portion 7, it is possible to accurately perform the treatment by observing the treatment target A from different directions without moving the treatment tool, which is an advantage of this embodiment.

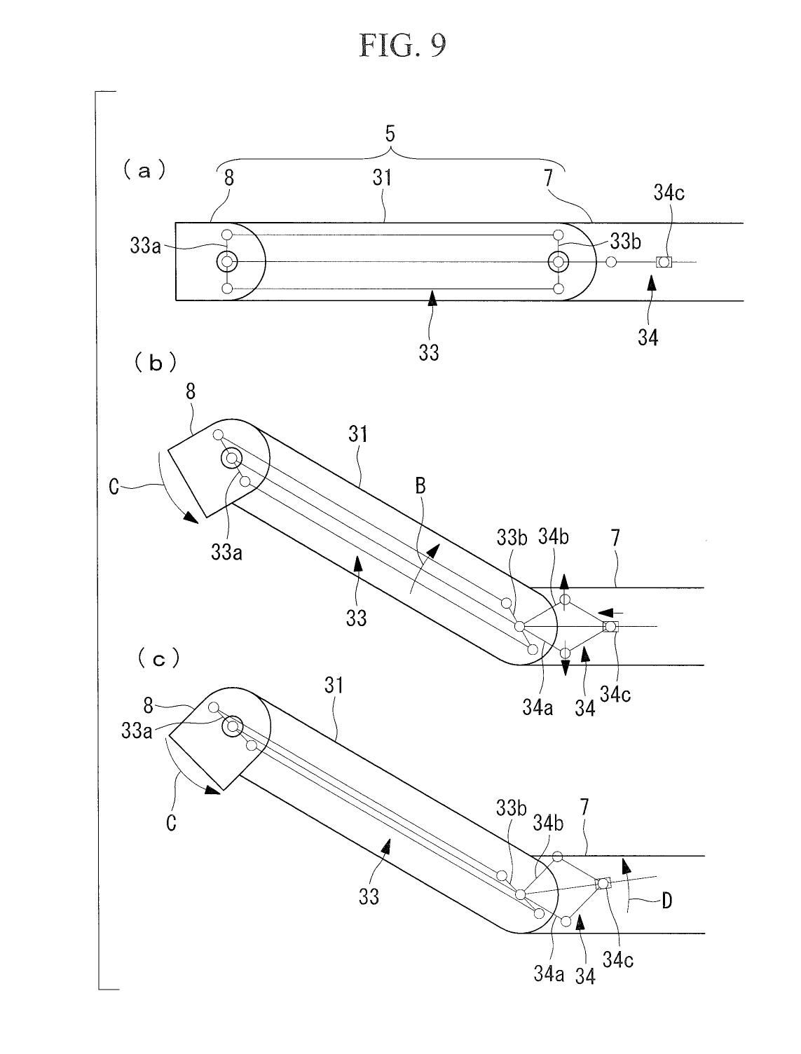

Note that, in this embodiment, although the motors 22 and 32 are employed as the distal-end-side rotating mechanism and the base-end-side rotating mechanism, respectively, and the interlinking mechanism is configured using the control unit 3, as shown in FIG. 9, pivoting of the distal end portion 8 and pivoting of the intermediate portion 31 may be performed in an interlinked fashion by employing a four-joint parallel linkage mechanism 33 as the interlinking mechanism.

This four-joint parallel linkage 33 is provided with a lever 34a that is pivotably connected to substantially the center of two opposing linkages 33a and 33b, and a four-joint slider linkage mechanism 34 in which this lever 34a serves as one linkage is provided. In addition, another linkage 34b of the four-joint slider linkage mechanism 34 is fixed to the linkage 33b which is one of the linkages in the four-joint parallel linkage 33 so that the linkage 34b is perpendicular to the linkage 33b.

When a slider 34c is slid toward the distal end from the state in which the proximal end portion 7, the intermediate portion 31, and the distal end portion 8 are extended straight, as shown in FIG. 9(a), the lever 34a forming one of the linkages in the four-joint slider linkage mechanism 34 is pivoted, as shown in FIG. 9(b), which causes the intermediate portion 31 to be pivoted relative to the proximal end portion 7 in the direction indicated by the arrow B and causes the distal end portion 8 to be pivoted relative to the intermediate portion 31 in the opposite direction (direction indicated by the arrow C).

By doing so, it is possible to perform pivoting of the intermediate portion 31 and pivoting of the distal end portion 8 in an interlinked fashion.

Note that, in the case in which the distal end portion 8 needs to be pivoted while keeping the intermediate portion 31 fixed, as shown in FIG. 9(c), the angle of the linkage 33b of the four-joint parallel linkage mechanism 33 to which the linkage 34b is connected may be changed by changing the angle of the four-joint slider linkage mechanism 34 itself in the direction indicated by the arrow D, and thereby the distal end portion 8 alone may independently be pivoted in the direction indicated by the arrow C in this way.

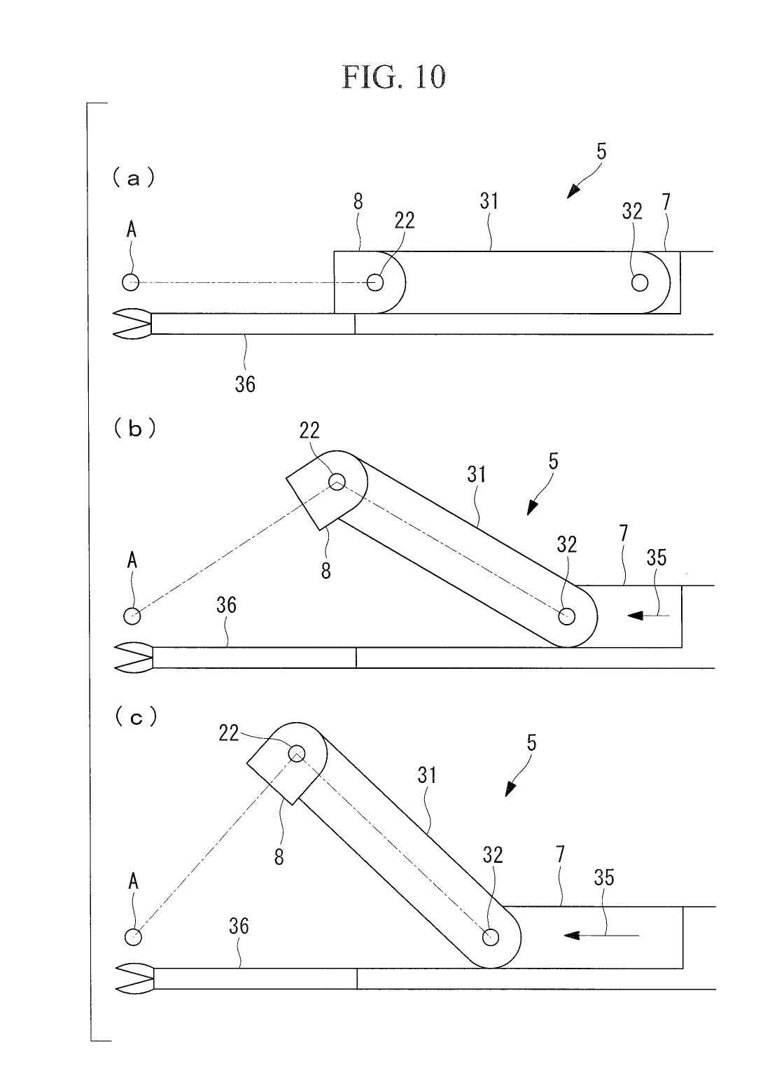

In addition, in the case in which the insert portion 5 is curved in a substantially S shape by means of the two rotating mechanisms 22 and 32, as shown in FIG. 10(a) to (c), in order to prevent the distance from the treatment target A to the lens groups 13 and 14 from changing, a moving mechanism 35 that moves the distal end portion 8 and the intermediate portion 31 in the longitudinal direction of the proximal end portion 7 may be employed. The moving mechanism 35 is, for example, a mechanism for sliding the proximal end portion 7 in the longitudinal direction that is parallel to the upper surface of a treatment tool 36, which is provided in a housing.

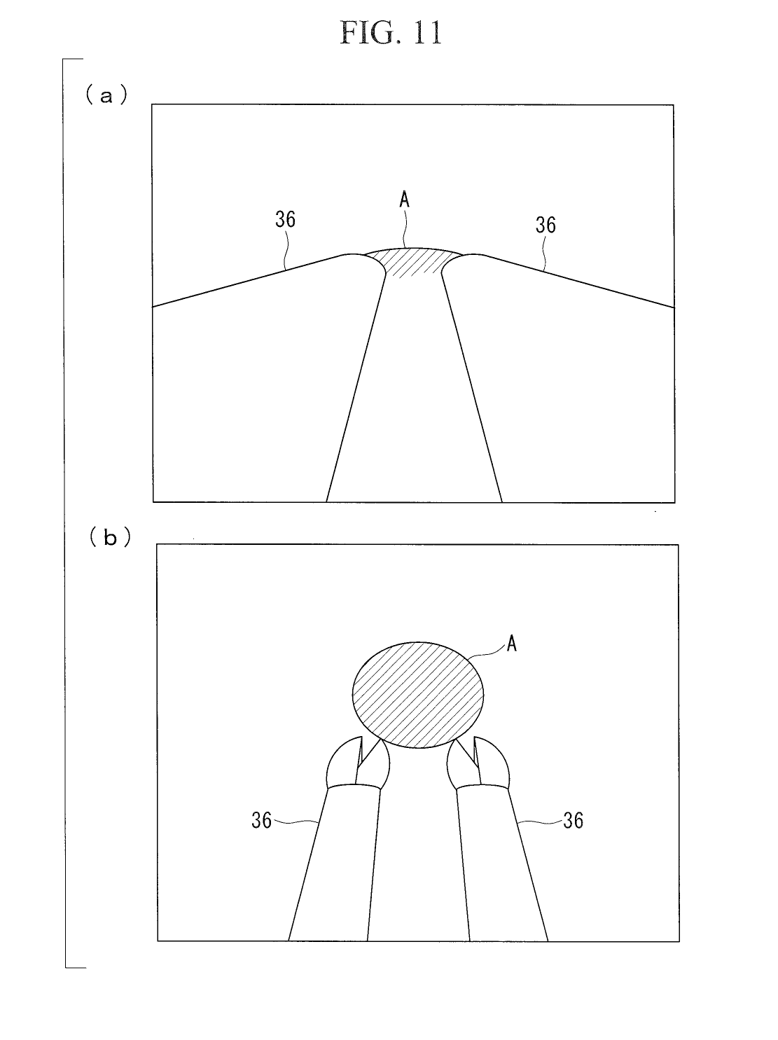

By doing so, even if the distal end portion 8 is moved in the direction away from the treatment target A by pivoting the intermediate portion 31, the distance from the treatment target A to the distal end portion 8 can be prevented from changing by linearly moving the distal end portion 8 and the intermediate portion 31 by means of the moving mechanism 35 in the direction that brings them close to the treatment target A. As a result, as shown in FIGS. 11(a) and (b), the treatment target A can be observed from an angle at which the treatment tool 36 does not become an obstacle by changing the observation angle for the treatment target A. In addition, there is an advantage in that, even if the observation angle for the treatment target A is changed, it is possible to always acquire well-focused screen images and to clearly observe the object.

The following inventions are derived from the aforementioned embodiment.

An aspect of the present invention provides a three-dimensional endoscope including the following elements: an optical system that is provided with a pair of lens groups which focus light coming from an object and which have substantially parallel optical axes, a pair of first prisms which deflect rays of light focused by the respective lens groups by 90° in opposite directions, and a pair of second prisms which deflect the rays of light deflected by the respective first prisms by an additional 90° so as to make the rays of light parallel to each other, and thereby forming two images exhibiting parallax; a distal-end-side rotating mechanism that rotates, as a single unit, the pair of lens groups and the pair of first prisms of the optical system relative to the pair of second prisms about an axis perpendicular to the optical axes; an imaging device that captures at a single imaging surface the two images exhibiting parallax formed by the optical system; and an image processing portion that rotates the two images acquired by the imaging device in opposite directions from each other by an angle formed between a plane containing the optical axes of the pair of lens groups and an axis perpendicular to the imaging surface by means of processing the images acquired by the imaging device.

With this aspect, the light coming from the object is focused by the pair of lens groups, and after the rays of light focused by the respective lens groups are deflected by 90° by the pair of first prisms, the rays of light are deflected by an additional 90° by the pair of second prisms and are captured to form two images exhibiting parallax at the single imaging surface of the imaging device. In addition, when the distal-end-side rotating mechanism is operated, the pair of lens groups and the pair of first prisms are rotated as a single unit relative to the pair of second prisms, thus changing the direction of the optical axes of the pair of lens groups, which makes it possible to observe the object from various angles.

In this case, when the lens groups and the first prisms are rotated by the distal-end-side rotating mechanism, the angles of the images formed at the imaging surface of the imaging device change in accordance with the rotation angle. Because the pair of first prisms deflect the rays of light focused by the respective lens groups in opposite directions, the angles of the two images at the imaging surface are also rotated in opposite directions. Therefore, the angles of the two images can be matched by rotating them, by means of the image processing portion, in opposite directions from each other by an angle formed between the plane containing the optical axes of the pair of lens groups and an axis perpendicular to the imaging surface, and a three-dimensional view can easily be made.

The above-described aspect may be provided with the following elements: a distal end portion that includes the pair of lens groups and the pair of first prisms; a long proximal end portion that is disposed at a proximal end side; an intermediate portion that is disposed between the proximal end portion and the distal end portion and that accommodates the second prisms and the imaging device; a base-end-side rotating mechanism that rotates the intermediate portion relative to the proximal end portion about an axis that is substantially parallel to an axis of the distal-end-side rotating mechanism; and an interlinking mechanism that operate the base-end-side rotating mechanism and the distal-end-side rotating mechanism in an interlinked fashion so that a ratio between an angle by which the intermediate portion is rotated by the base-end-side rotating mechanism relative to the proximal end portion and an angle by which the distal end portion is rotated by the distal-end-side rotating mechanism relative to the proximal end portion is kept constant.

By doing so, when the intermediate portion is rotated relative to the proximal end portion by operating the base-end-side rotating mechanism, the distal-end-side rotating mechanism is rotated in the opposite direction by the operation of the interlinking mechanism, and thus, the whole unit forms a substantially S-shaped curve. At this time, because this interlinking occurs so that the ratio between the rotation angle of the intermediate portion relative to the proximal end portion and the rotation angle of the distal end portion relative to the proximal end portion is kept substantially constant, when the rotation angle of the intermediate portion is increased, the rotation angle of the distal end portion is also increased, and therefore the same object can easily be observed from various angles.

In addition, the above-described aspect may be provided with a moving mechanism that moves the intermediate portion and the distal end portion in a longitudinal direction of the proximal end portion so that a distance between the pair of lens groups and the object is prevented from being changed when the distal-end-side rotating mechanism and the base-end-side rotating mechanism are operated in the interlinked fashion by the interlinking mechanism.

By doing so, even if the rotation angle of the intermediate portion is changed, the distance between the lens groups in the distal end portion and the object can be prevented from being changed by moving the intermediate portion and the distal end portion in the longitudinal direction of the proximal end portion by operating the moving mechanism, and thus, the same object can clearly be observed from various angles.

The above described embodiments achieve an effect in which interference with organs or the like in the surroundings can be decreased by keeping the rotation radius minimum and the maneuverability can be enhanced.

1. A three-dimensional endoscope comprising:
an optical system that is provided with a pair of lens groups which focus light coming from an object and which have substantially parallel optical axes, a pair of first prisms which deflect rays of light focused by the respective lens groups by 90° in opposite directions, and a pair of second prisms which deflect the rays of light deflected by the respective first prisms by an additional 90° so as to make the rays of light parallel to each other, and thereby forming two images exhibiting parallax;
a distal-end-side rotating mechanism that rotates, as a single unit, the pair of lens groups and the pair of first prisms of the optical system relative to the pair of second prisms about an axis perpendicular to the optical axes;
an imaging device that captures at a single imaging surface the two images exhibiting parallax formed by the optical system; and
an image processing portion that rotates the two images acquired by the imaging device in opposite directions from each other by an angle formed between a plane containing the optical axes of the pair of lens groups and an axis perpendicular to the imaging surface by means of processing the images acquired by the imaging device.

2. The three-dimensional endoscope according to claim 1, further comprising:
a distal end portion that includes the pair of lens groups and the pair of first prisms;
a long proximal end portion that is disposed at a proximal end side;
an intermediate portion that is disposed between the proximal end portion and the distal end portion and that accommodates the second prisms and the imaging device;
a base-end-side rotating mechanism that rotates the intermediate portion relative to the proximal end portion about an axis that is substantially parallel to an axis of the distal-end-side rotating mechanism; and
an interlinking mechanism that operate the base-end-side rotating mechanism and the distal-end-side rotating mechanism in an interlinked fashion so that a ratio between an angle by which the intermediate portion is rotated by the base-end-side rotating mechanism relative to the proximal end portion and an angle by which the distal end portion is rotated by the distal-end-side rotating mechanism relative to the proximal end portion is kept constant.

3. The three-dimensional endoscope according to claim 2, further comprising:
a moving mechanism that moves the intermediate portion and the distal end portion in a longitudinal direction of the proximal end portion so that a distance between the pair of lens groups and the object is prevented from being changed when the distal-end-side rotating mechanism and the base-end-side rotating mechanism are operated in the interlinked fashion by the interlinking mechanism.
